(19) United States
(12) Patent Application Publication
(10) Pub. No.: US 2004/0137478 A1
(43) Pub. Date: Jul. 15, 2004

(54) Enhancing the sensitivity of a microsphere sensor

(76) Inventors: Stephen Arnold, New York, NY (US); Iwao Teraoka, Rye, NY (US); Frank Vollmer, New York, NY (US)

Correspondence Address: STRAUB & POKOTYLO, 620 TINTON AVENUE, BLDG. B, 2ND FLOOR, TINTON FALLS, NJ, 07724, US

(21) Appl. No.: 10/690,979

(22) Filed: Oct. 22, 2003

Related U.S. Application Data
(60) Provisional application No. 60/420,436, filed on Oct. 22, 2002.

(51) Int. Cl. C12Q 1/68; G01N 33/53; C12M 1/34

(52) U.S. Cl. 435/6; 435/7.1; 435/287.2

(57) ABSTRACT

Microsphere sensors (i) having receptors selectively substantially provided at only an equator region, (ii) formed of a relative high IR material, and/or (iii) having a relatively small radius are provided with improved sensitivity. Such a microsphere sensor may be made by selectively treating an equator region of the microsphere forming a small concentrated receptor band on the high sensitivity portion of the microsphere surface. Changing the selected laser frequency applied to the microsphere sensor to a shorter wavelength also improves sensitivity. Physical properties of the microsphere sensor system: index of refraction, laser frequency, and microsphere radius may be adjusted in concert to match the target entity molecule size. These improvements in sensitivity may allow detection and/or identification of unknown target entities based on detectable step shifts observable in light modes due to the adsorption of even a single molecule as small as about 200,000 Da.

### RELATED APPLICATION

This application claims benefit to U.S. Provisional Application Serial No. 60/420,436, titled “ENHANCING THE SENSITIVITY OF A MICROSPHERE SENSOR USING A SHIFT OF WHISPERING GALLERY MODES IN THE MICROSPHERE CAUSED BY ADSORPTION OF TARGET ENTITIES”, filed on Oct. 22, 2002, and listing Stephen Arnold, Iwao Teraoka, and Frank Vollmer as the inventors. That application is expressly incorporated herein by reference. The scope of the present invention is not limited to any requirements of the specific embodiments in that application.

### FEDERAL FUNDING

This invention was made with Government support and the Government may have certain rights in the invention as provided for by grant number BES-0119273 awarded by the National Science Foundation.

### FIELD OF THE INVENTION

The present invention concerns detecting the presence of, identifying the composition of, and/or measuring an amount or concentration of substances, such as chemical or biological entities, even in amounts as small as single proteins or virus particles. More specifically, the present invention, concerns methods and apparatus to enhance the sensitivity of a microsphere sensor system that use whispering gallery modes (WGMs), as well as enhancing the sensitivity of the microsphere sensors themselves.

### BACKGROUND

In the recent past, the need for sensors for detecting infectious agents, toxins, and the like has taken on added urgency as the world anticipates bio-terrorism. There is also a need to detect small amounts of proteins, DNA, and the like for various reasons. One known device used to detect the presence of small particles is a microsphere sensor coupled to an optical carrier, e.g., an eroded optical fiber, one end of which is optically coupled with a light source and the other end with a light detector. Whispering gallery modes of the light circulating around the microsphere can be observed in optical signals detected at the detector. Particles adsorbed on the surface of the microsphere may shift the whispering gallery modes.

U.S. patent application Ser. No. 10/096,333, filed Mar. 12, 2002, titled “DETECTING AND/OR MEASURING A SUBSTANCE BASED ON A RESONANCE SHIFT OF PHOTONS ORBITING WITHIN A MICROSPHERE”, and listing Stephen Arnold and Iwao Teraoka as the inventors (referred to as “the '333 application”) describes such microsphere sensors, as well as their use and manufacture. That application is incorporated herein by reference. Known microsphere sensors, like the ones described in the '333 application, may be useful for detecting the presence and/or amount of small particles; however, these known sensors may have limits on the minimum size of the particles that may be detected and/or identified. In addition, known microsphere sensors and detection/identification methods are directed to detection/identification based on a number of same composition particles affecting the microsphere sensor.

Increasing the sensitivity of such sensors would be useful and could expand potential applications for such sensors, especially if their sensitivity could be improved to the point where an individual protein molecule, virus particle, or other small entity could be detected and identified. For example, enhancing sensor sensitivity could facilitate (i) detections of smaller size particles, (ii) detections of extremely low concentration exposures that might otherwise go unnoticed, (iii) earlier warnings to exposures, (iv) a greater area of overall coverage with fewer sensors, etc.

In light of the above discussion, it is clear that there is a need to improve microsphere sensor sensitivity.

### SUMMARY OF THE INVENTION

The invention may be used to enhance the sensitivity of a microsphere sensor and/or a microsphere sensor system by selectively promoting adsorption of target entities (ligands) in the identified high sensitivity region near the equator of the microsphere. The invention may accomplish this by using a microsphere specially treated or silanized in the equator region to create a band (e.g., a narrow band) of receptors. The invention describes methods for obtaining a microsphere with receptors substantially limited to a highly sensitive region near the equator of the microsphere.

Alternately, or in addition, the invention may improve sensitivity by changing the selected frequency of the laser light used in a microsphere detection system to the blue light region of the spectrum, e.g. approximately 400 nm, to reduce the size of a detectable change due to an adsorbed particle. The wavelength, λ, may be selected to match the characteristics of the microsphere. A ratio may exist between the size of the microsphere and the size of the wavelength used such that a reduction in the size of the microsphere may correspond to a reduction in the size of the selected wavelength, λ, for example, to ensure that a resonance occurs in the microsphere.

Alternately, or in addition, the invention may use a microsphere of a material having a higher refractive index than that of silica (which has a refractive index of about 1.47) to further reduce the size of a detectable change due to an adsorbed particle. In one embodiment of the invention, the microsphere may have a refractive index of about 1.7. In such an embodiment, the microsphere may be formed of amorphous sapphire. Alternately, or in addition, to further increase sensitivity, the size of the microsphere may be reduced from a radius of approximately 75 micrometers to a radius of approximately 3.6 to 10 micrometers.

In various embodiments of the invention, one or more of the enhancements to the microsphere sensor and sensor system may be employed to increase sensitivity for applications presently using known microsphere sensors and to facilitate microsphere sensor use in new applications which would not have been possible or practical using the lower sensitivity microsphere sensors.

One embodiment of the invention may combine a microsphere that has been selectively treated to adsorb target (or unknown) entities in a high sensitivity region, a reduced microsphere size, use of blue light frequency (shorter wavelength), and an increased index of refraction microsphere to enable detecting a single protein molecule such as thyroglobulin, ferritin, and virus particles, e.g., lamda phage.

The invention also describes methods for fabricating microsphere sensors having receptors substantially only at a sensitive equator region. One embodiment of sensor fabrication includes: (i) selecting a microsphere with properties (IR and radius) suited to the intended sensing application, (ii) optically coupling an eroded optical fiber with the microsphere at an equator, (iii) coating the microsphere with a UV reactive binding agent, such as an epoxy, (iv) selectively establishing an equator region with receptor material by immersing the microsphere in a solution with receptors, (e.g., of selected amines) and irradiating the equator band with UV light coupled into the microsphere through the eroded optical fiber causing a reaction between the receptors in the solution and the binding agent, (v) washing the resulting sphere, and (vi) establishing the non-equator region as a non-interacting region (e.g., by immersing the microsphere in a solution of mono-secondary amines, irradiating the entire surface with UV light (e.g., from an external lamp) causing a reaction between the mono-secondary amines and any un-reacted binding agent, and washing).

### BRIEF DESCRIPTION OF THE FIGURES

FIG. 1 illustrates an exemplary microsphere sensor of the present invention.

FIG. 2 is a bubble chart illustrating exemplary operations that may be used to fabricate a microsphere sensor, such as the microsphere sensor shown in FIG. 1, in accordance with the invention.

FIGS. 3 and 4 are flow diagrams describing exemplary methods that may be used to perform the operations of FIG. 2, in accordance with the invention.

FIG. 5 is a flowchart illustrating an exemplary method that may be used to fabricate a microsphere sensor, such as the microsphere sensor shown in FIG. 1, in accordance with the present invention.

FIG. 6 is a block diagram of an exemplary detection system implemented in accordance with the present invention.

FIG. 7 illustrates an exemplary detection system implemented in accordance with the present invention.

FIG. 8 is a more detailed illustration of a microsphere sensor, in accordance with the present invention, submerged in an aqueous medium, with a target entity captured on a receptor of the receptor band.

FIG. 9 is a flow chart illustrating an exemplary method that may be used to detect and/or identify a single protein molecule or other small target entity in accordance with the invention.

FIG. 10 is a detailed illustration of a microsphere submerged in an aqueous medium and attached to an eroded optical fiber.

FIG. 11 shows saturation shifts of WGM resonances measured for BSA protein adsorption as a function or microsphere radius.

### DETAILED DESCRIPTION

Section 1: Enhanced Microsphere Sensor

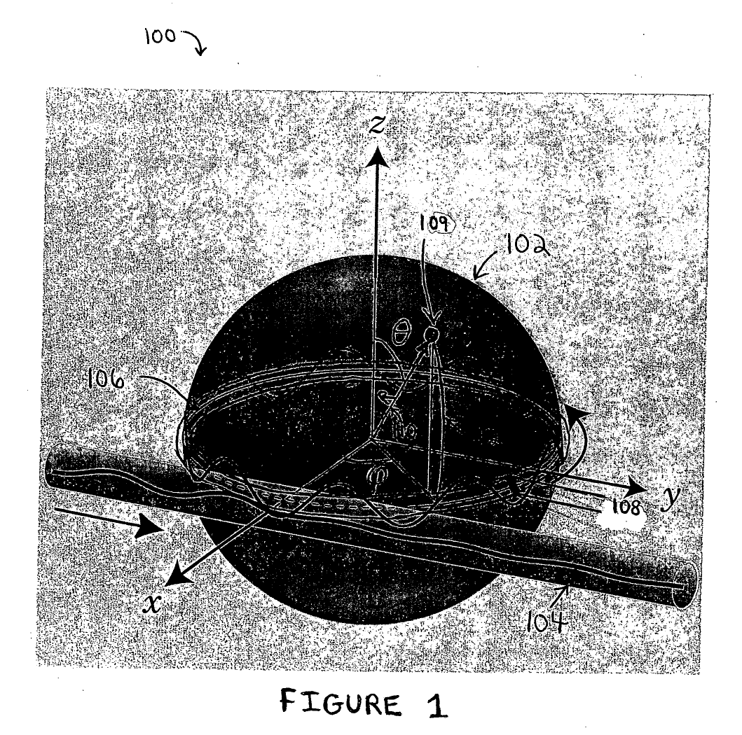

FIG. 1 is an illustration of a microsphere sensor 100 in accordance with the invention. The microsphere 102 is optically coupled with an eroded optical fiber 104 at a point or segment on the equator 106 of the microsphere 102. In accordance with the invention, a (narrow) region or band of target entity receptor material 108 has been selectively formed on the equator 106 of the microsphere 102. The target region 108 (determined to be an extremely sensitive region for adsorption of a single protein or other small target entity) has been selectively treated to promote adsorption, e.g., by silanizing the area. This feature of the invention, of a limited highly sensitive receptor band 108 including the equator 106 is in contrast to known microsphere sensors which may have receptor material on the entire surface of the microsphere, or over large regions of the surface. In known microsphere sensors, the position and size of the receptor location is not required to be tightly controlled. In such known microsphere sensors sensitivity to each captured target entity may vary depending on the location rᵢ 109 on the microsphere surface that the target entity is captured by the receptor. In such cases, the microsphere sensor may require that a large number of target entities be captured by the receptors to detect, quantify, and/or identify a target (or unknown) substance. By restricting receptors to the high sensitivity area 108, where the level of change caused by a single target entity is identifiable and relatively uniform in magnitude, the invention can be used to make each target entity captured by a receptor significant and allow for detection and/or identification based on a single particle.

A frequency shift in a resonance mode, due to the adsorption of a target (or unknown) entity, in the high sensitivity receptor band 108, may be detected by a detector optically coupled with the microsphere sensor 100. For microsphere sensors 100 provided with the high sensitivity receptor band 108, the level of frequency shift, due to the adsorption of a target entity, may vary (approximately) as 1/R^{5/2}, where R 110 is the radius of the microsphere 102.

In accordance with the invention, the sensitivity of the microsphere 102 may be further increased by reducing the size of the microsphere 102. That is, microspheres with a radius of approximately 75-300 μm are common. The invention may provide a microsphere with a radius of approximately 3.6-10 μm. In accordance with the invention, the microsphere's sensitivity may be further increased by changing the index of refraction of the material used in the microsphere 102 to a material with a higher refractive index. That is silica microspheres having a refractive index of 1.47 are common. The invention may provide microspheres of an alternative material, e.g., amorphous sapphire, with refractive index 1.7.

The index of refraction (IR) selected for the microsphere 102 and the range of radii 110 of the microsphere 102 can be matched to the target entity or group of target entities which the microsphere sensor 100 is intended to detect and/or identify. For a high refractive index material, e.g., amorphous sapphire, the sphere radius is preferably between 3.6 to 10 μm when it is desired to detect 1 molecule of a target entity of approximately 200,000 Da. For larger target entities, the radius 110 could be increased in inverse proportion to the molecular weight of the target. For large targets, e.g., target entities or molecules of several million Da, a material with a relatively lower refractive index could be selected for the microsphere 102. For example, using a silica microsphere 102, which is a material used in known microspheres, with an index of refraction=1.47 in water, large target entities may be detected; however, the minimum size of the silica microsphere is limited to a radius 110 of approximately 75 micro-meters. In contrast, using amorphous sapphire microspheres (having a refractive index=1.7 in water), allows the size of the microsphere 102 to be reduced to a radius 110 of approximately 3.6 micro-meters allowing smaller size target entity molecules to be detected.

Section 2: Sensor Fabrication:

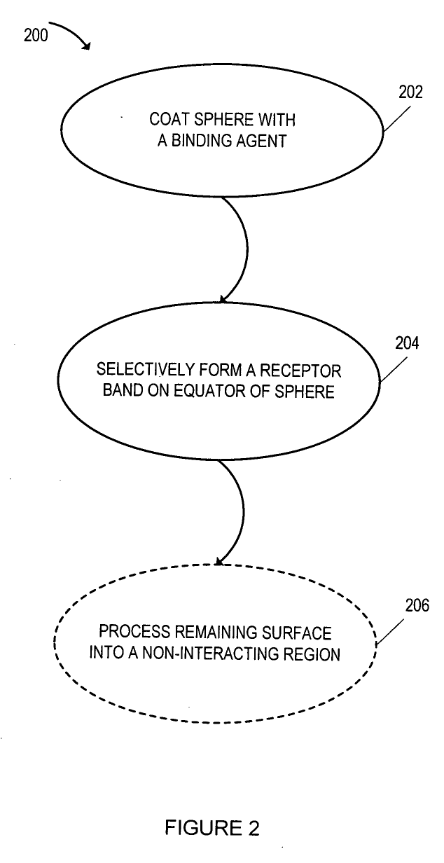

FIG. 2 shows a bubble chart 200 illustrating exemplary operations that may be performed in fabricating a sensor in accordance with the present invention. Basically two or three operations may be performed to obtain a receptor band limited to the high sensitivity equator region. First operation 202 involves coating the sphere's surface with a reactive binding agent, such as an epoxy or adhesive material. A second operation 204 involves selectively forming a receptor band on the equator of the microsphere. An optional third operation 206 involves processing the remaining surface of the microsphere into a non-interacting region.

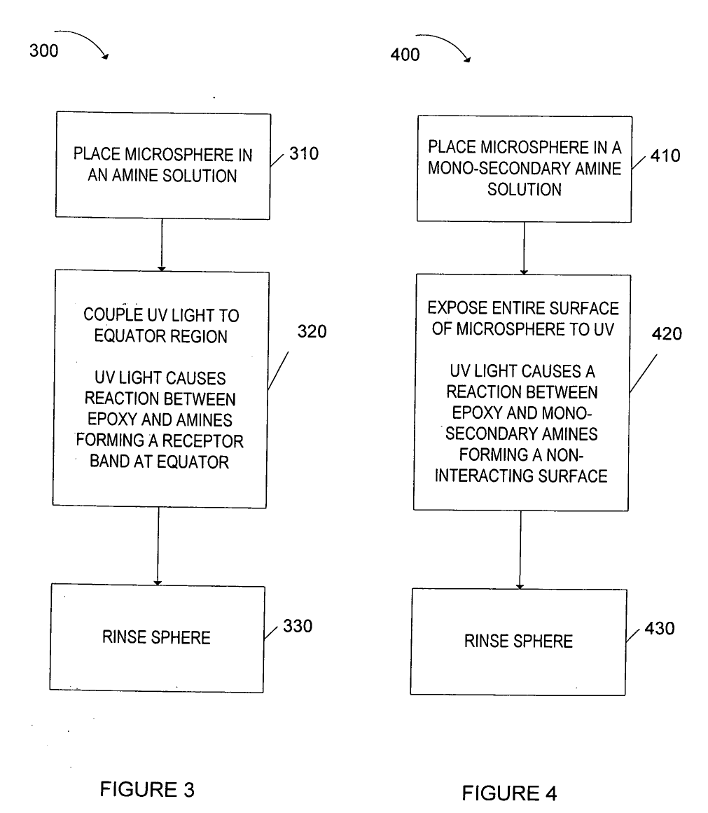

FIG. 3 is a flow diagram 300 of an exemplary method 300 that may be used to perform operation 204, i.e., selectively forming a functional receptor band on the equator. In step 310, the microsphere, covered in epoxy (or some other binding agent), is placed in a solution of amines (or some other receptor). In step 320, UV light is coupled into the equator region of the microsphere via an eroded optical fiber. The UV light causes a reaction between the epoxy and the amines creating a receptor band for target entities on the equator region. In step 330, the microsphere may be washed or rinsed off to remove any un-reacted amines from the microsphere surface. The microsphere now has a receptor band at the equator region, but may have un-reacted epoxy remaining on the rest of the sphere's surface.

FIG. 4 is a flow diagram of an exemplary method 400 that may be used to perform operation 206, i.e., processing the remaining surface into a non-interacting region. In step 410, the microsphere, with un-reacted epoxy on the non-equator region, is placed in a solution of mono-secondary amines. In step 420, the entire surface of the sphere is exposed to UV light, such as from an external source. The UV light causes a reaction between the epoxy and the mono-secondary amines creating a non-interacting coating on the non-equator region. In step 430, the microsphere may be washed or rinsed off to remove any un-reacted mono-secondary amines from the microsphere surface. The microsphere 102 now has a receptor band at the equator region 108 and a non-interacting region on the rest of the sphere's surface.

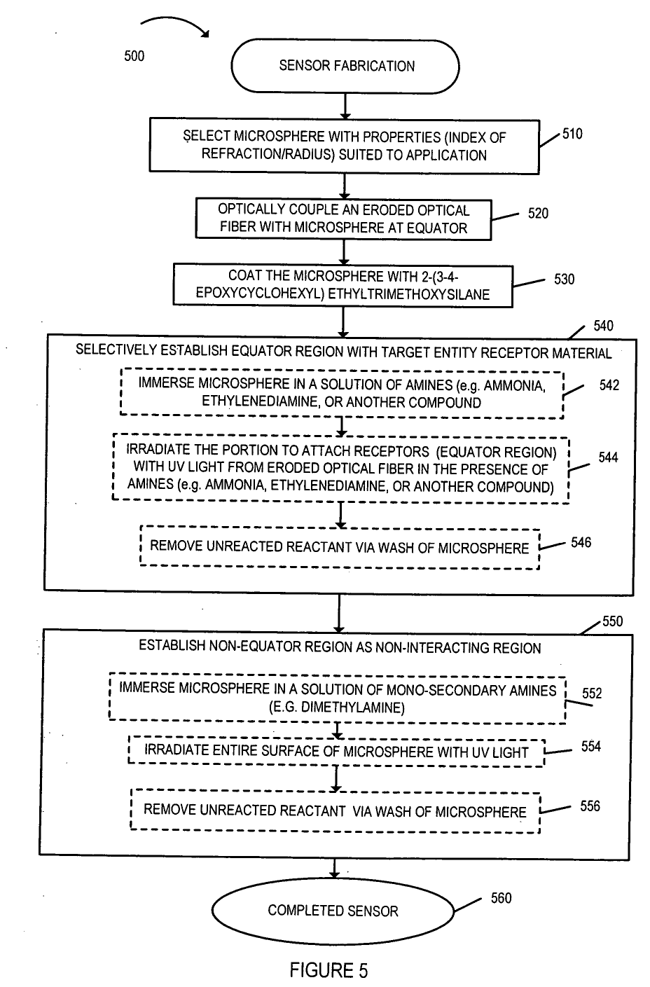

FIG. 5 is a flowchart 500 illustrating an exemplary microsphere sensor 100 fabrication process of the present invention. In step 510, a microsphere 102 is selected with properties suitable for the intended application. The radius 110 of the microsphere 102 and/or material composition of the microsphere 102 (e.g., having a desired index of refraction) may vary depending on the application, as described above.

In step 520, an eroded optical fiber 104 is optically coupled to the microsphere. For example, an eroded optical fiber 104 may be pressed up to the equator 106 of the microsphere 102 and attached using a polymer cement, e.g. Cytop. Cytop, a product of the Asahi glass company, which is a fluorinated polymer with the same refractive index as water. Other similar cements with low refractive indices may also be used.

In step 530, the microsphere 102 is coated with 2-(3-4-epoxycyclohexyl) ethyltrimethoxysilane or a similar compound. The 2-(3-4-epoxyxcyclohexyl) ethyltrimethoxysilane is an exemplary epoxy that may be used to adhere amines to the surface of the microsphere under the presence of UV light or heat. In some embodiments, the coating of the microsphere 102 (step 530) may be performed before coupling the microsphere 102 and the eroded optical fiber 104 (step 520).

In step 540, the equator region 108 is selectively established with target entity receptor material. The target entity receptor material may be an amine such as ammonia, ethylenediamine, or another similar compound. The specific amine selected will be complementary to the specific protein or other target entity for which the sensor is designed. In sub-step 542, the microsphere 102 may be immersed in a solution containing the amines or may be exposed to the amines via gas phase. In sub-step 544, the portion of the microsphere surface to be established with amines is irradiated with UV light in the presence the amines, e.g., ammonia, ethylenediamine, or another similar selected compound. The portion to be established is band region including the equator, otherwise referred to as the high sensitivity target region 108. The band region 108, defined by photon orbit, may be written (established) by setting up a resonance with UV laser light transmitted through the eroded optical fiber 104 into the microsphere 102 causing a reaction between the epoxy and the amines resulting in the establishment of receptor material in the band region 108. Thus, as can be appreciated from the foregoing, an orbit used in detection may be the same as the orbit used in fabrication. The angle from the equator for an orbit is approximately (1 μL)^{1/2} where L is the angular momentum quantum number. This translates into an arclength (in region 108) on the microsphere surface of approximately [Rλ/(2πn)]^{1/2}, where λ is the wavelength of the laser light used and n is the refractive index. For an exemplary microsphere radius of 100 micro-meters, n=1.47, and λ=1.3 micro-meters, the arc length, defining the equator region 108, is approximately 3 micrometers. Depending on the target entity, the size of the receptor band 108 at the equator 106 may be controlled by selecting the laser light frequency and controlling the laser light transmitted into the eroded fiber 104 used to establish the resonance. In sub step 546, the un-reacted reactant (the amines described in step 540 on the surface of the microsphere, which were not exposed to the UV light from the fiber) are removed from the microsphere surface. This may be accomplished by a washing or rinsing of the microsphere. At this point, the microsphere has a band of selected receptor material 108 established and secured on the equator 106 of the microsphere 102. The rest of the surface of the microsphere may still contain epoxy which may react with various amines when exposed to UV light or heat.

In step 550, the non-equator region is established as a non-interacting region. In sub-step 552, the microsphere 102 may be immersed in a solution containing mono-secondary amines or may be exposed to a mono-secondary amines via gas phase. Two examples of mono-secondary amines are dimethylamine and diethylamine. In sub-step 554, the entire surface of the microsphere 102 may be irradiated with UV light in the presence of a mono-secondary amine or another similar compound. The UV light may be sourced from an external UV lamp. Alternately, instead of applying UV light to the microsphere, the microsphere may be heated. This application of UV light or heat causes a reaction between any epoxy in the non-equator region with the mono-secondary amines causing the mono-secondary amines to adhere to the epoxy. The mono-secondary amines, adhered to the epoxy surface of the microsphere will not act as complementary receptors for protein molecules. Thus, step 554 has rendered the region outside the equator receptor region 108, a non-interacting region. In sub-step 556, the microsphere 102 can be rinsed to remove any un-reacted mono-secondary amines.

The process ends at node 560, with a completed microsphere sensor 100, which has a highly sensitive target receptor region 108 at the equator 106, and a non-interacting region covering the remainder of the surface of the microsphere 102, in accordance with the invention.

Naturally, other sub-steps can be used. Further, other materials may be used (e.g., depending on the target entity to be sensed).

Section 3: Sensor Use (System Apparatus and Method of Implementation)

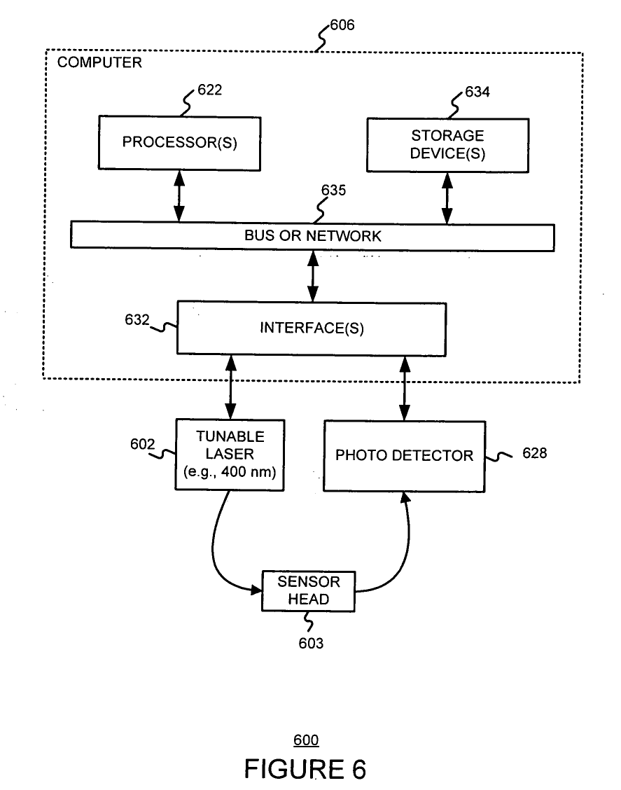

FIG. 6 shows a block diagram of exemplary sensor detection system 600, implemented in accordance with the present invention, that may be used for the detection and/or identification of substances such as biomolecules, e.g. proteins or virus particles. In accordance with the invention, the sensitivity of the sensor detection system 600 has been enhanced over known systems such that single protein or other small entity detection and identification is possible.

Sensor detection system 600 may include a laser, such as a tunable Distributed Feedback Laser (DFB) 602, a sensor head 603, an optical detector, e.g., a photo detector 628, and a computer 606. Computer 606, includes a processor 622, storage device(s) 634, e.g., memory, interface(s) 632, and a bus or network 635 over which the various elements may interchange data and information. The tunable laser 602 may emit light into or through a sensor head 603. Photo detector 628 may detect light from the sensor head 603. The evaluation of changes in signal output from photo detector 628 may be used to determine the existence of, or the amount of, a substance being sensed (target entity or entity) by the sensor head 603. In systems 600 including a computer 606, the processor 622 under the direction of routines in memory 634, may control the laser 602 through interface 632. The processor 632 may receive output signaling from photo detector 628 through interface 622 and process the signaling to determine the existence, or amount of a substance being sensed by the sensor head 603. Sensor head 603 may have any of a number of possible configurations including a single microsphere sensing head, a multiple microsphere sensing head using different receptors on different spheres, and a multiple microsphere sensing head including at least one microsphere without receptors to be used to characterize and remove common mode noise. (See, e.g., the '333 application.)

In some embodiments of the invention, the sensor detection system 600 may be implemented using one or more modules. Such modules may be implemented using software, hardware, or a combination of software and hardware.

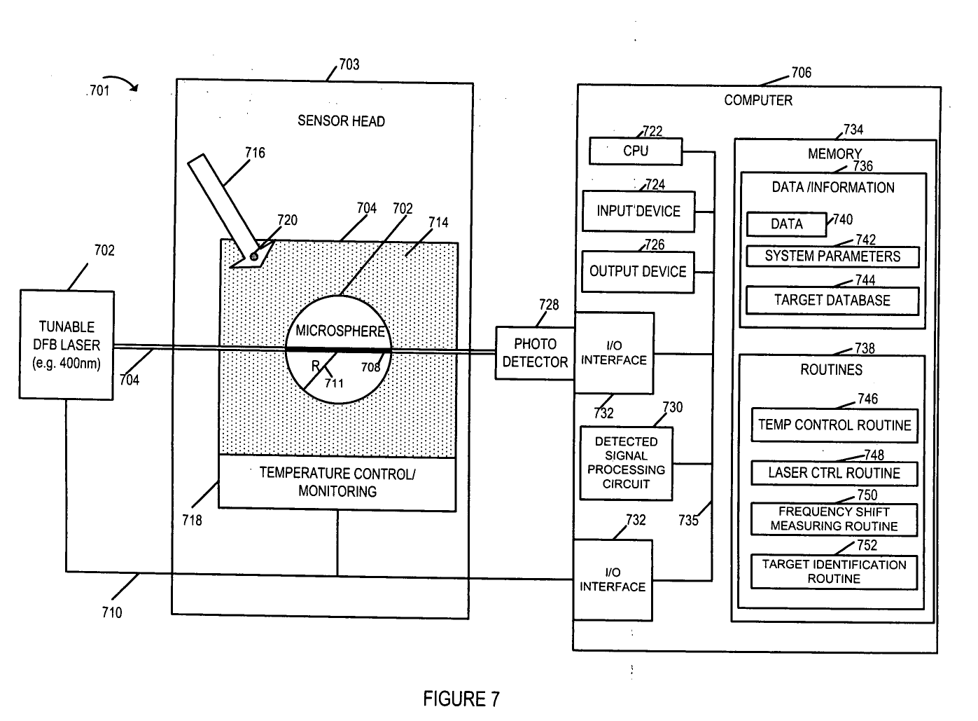

FIG. 7 illustrates an exemplary sensor detection system 701 which may be one possible exemplary embodiment of system 600. Sensor detection system 701 may include a laser, such as a tunable Distributed Feedback Laser (DFB) 702, a sensor head 703 including a microsphere containment vessel 704, and an optical detector, e.g., a photo detector 728 which may be coupled to a computer 706 through I/O interface 732. The tunable DFB laser 702 may be, e.g., a blue diode laser with external cavity operating at a wavelength of about 400 nm. The laser 702 selected for system 701 operates at a wavelength of about 400 nm, is in contrast to the known microsphere systems using a nominal wavelength of 1.34 μm. This wavelength may be used in concert with other sensor design changes to reduce the size of the smallest detectable protein polarizability.

The microsphere containment vessel 704 may include a microsphere 702 including a receptor band 708, an aqueous medium 714, a target entity injection element 716, and a temperature control/monitoring device 718. Microsphere 702 may include one or more of the features described above. Target entity injection element 716, may hold and control the release of a sample including a target entity 720, e.g., a protein molecule. The target entity 720 may diffuse through the aqueous medium 714, e.g., water, to the microsphere's surface where it may be adsorbed in the receptor band 708, become polarized, and shift the frequency of the resonant modes. Temperature control/monitoring device 718 may include temperature sensors, heaters, and regulation circuitry, for reporting the temperature of the vessel 704, microsphere 702, and/or aqueous medium 714 to the computer system 706 and/or regulating the temperatures.

In some embodiments, multiple microspheres 702 may be used in the same aqueous medium 714. In some embodiments, multiple microsphere sensors, each sensor customized (with specific complementary receptors, specific physical characteristics, and a specific size receptor band) for detection of a specific target entity may be coupled with the detection system. In some embodiments, microspheres similar or identical to sensor microspheres, except without a target receptor material may be included. Those microspheres without target receptor material may provide information on resonance characteristics changes, due to environmental disturbances and may be used to characterize “common mode noise”.

In some embodiments, the microsphere 702 may be inserted and removed from the microsphere containment vessel 704. In some embodiments, adsorption of target entities onto the microsphere surface in receptor band 708 may occur while the microsphere 702 is removed from the aqueous medium 714, and the microsphere 702 may be inserted into the medium 714 for measurement purposes.

In some embodiments, the microsphere 702 sensor may not be situated in an aqueous medium 714, but rather in a gaseous medium, e.g., air. In some embodiments, microsphere 702 sensor may not be situated in a containment vessel 704, but rather may be placed in an open environment. In some embodiments, an injection element 716 may not be used. In some embodiments, gaseous or aqueous medium, which may contain target entities, may be directed or forced to pass over the microsphere sensor.

The photo detector 728 may provide data to a computer system 706 through I/O interface 732. In some embodiments the photo detector 728 may be included as part of the computer system 706. The computer system 706 may include a processor (e.g., a CPU) 722, an input device 724, an output device 726, a detected signal processing circuit 730, an I/O interface 732, and memory 734 coupled together via bus or network 735 over which the various elements may interchange data and information. Memory 736 may include data/information 736 and routines 738. Data/information 736 may include data 740, system parameters 742, and target database information 744. Routines 738 may include a temperature control routine 746, a laser control routine 748, a frequency shift measurement routine 750, and/or a target identification routine 752. The processor 722 may be used to execute the routines 738 and use the data/information 736 in memory 734 to detect and identify substances such as biomolecules, e.g., proteins or virus particles, etc. in accordance with the methods of the invention. The input device 724 may include keyboards, keypads, etc. and may be used to notify the computer system 706, that a target entity 720 has been released into aqueous medium 714. Output devices 726 may include displays, printers, speakers, etc. which may indicate temperature stabilization, prompts to release target entities 720, detected frequency shifts, and identified target entities 720.

The system 701 may operate as follows. Photo detector 728 receives the light transmission from the laser 702, which has been altered by the resonant modes of WGMs of microsphere 702 and shifts in resonant mode due to adsorbed target entities 720, and converts the optical signal to an electrical signal. Detected signal processing circuit 730 receives the electrical signal from the photo detector 728 and detects, e.g., such resonance modes (manifested as dips in the transmitted signal which correspond to resonant modes). I/O interface 732 may include line drivers and receivers, A/D converters, D/A converters, frequency counters, etc. Data 740 may include data collected on the transmitted signal, e.g., frequency, detected resonant modes, shifts detected in resonant modes, and temperature data of the microsphere 702 and/or aqueous medium 714. System parameters 742 may include frequency of the laser 702, radius 711 of the microsphere 702, parameters defining a specially treated target reception region 708 on the microsphere 702, stabilization temperature, index of refraction of the microsphere 702, index of refraction of the aqueous medium 714, thermal models, and calibration parameters associated with the system 701. Target database 744 may include look-up tables associating steps changes or level shifts in the frequency of the modes observed with specific target entities 720, e.g., protein molecules such as thyroglobulin, ferritin, or virus particles such as lambda phage. Temperature control routine 746 may forward temperature sensor information from temperature control/monitoring device 718, and may control circuitry within device 718 to maintain temperature stabilization of the microsphere 702 and/or aqueous medium 714 at pre-determined levels. Laser control routine 748 may control and monitor the tunable DFB laser 702 to maintain a detectable WGM signal at the photo detector 728 and provide current precise laser frequency information to the computer system 706. Frequency shift measuring routine 750 processes information from the detected signal processing circuit 730 to detect step changes of shifts in mode frequencies with time. Target identification routine 752 uses the output of the frequency shift measuring routine 750 to match the step level changes to a corresponding target entity, e.g., a specific protein molecule or virus particle such as a lambda phage virus particle.

The tunable laser 702 is optically coupled with the microsphere 702, and the photodetector 728 via an optical fiber 704. The optical fiber 704 is eroded at the attachment point to the microsphere 702. This allows light being transmitted from the laser 702 to the photodetector 728 to be coupled into a whispering gallery mode of the microsphere 702, create detectable resonant modes in the transmission, and create detectable frequency shifts in the resonant modes in response to adsorbed target entities on the microsphere 702. In other embodiments, the light from the laser 702 is coupled into the microsphere 702 via means other than an eroded optical fiber, e.g., via lenses, splitters, etc. Electrically, the laser 702 may be coupled to the temperature control/monitoring circuitry 718 of the microsphere containment vessel 704 and the I/O interface 732 of the computer system 706 via bus 710 over which measurement signals and control information is exchanged.

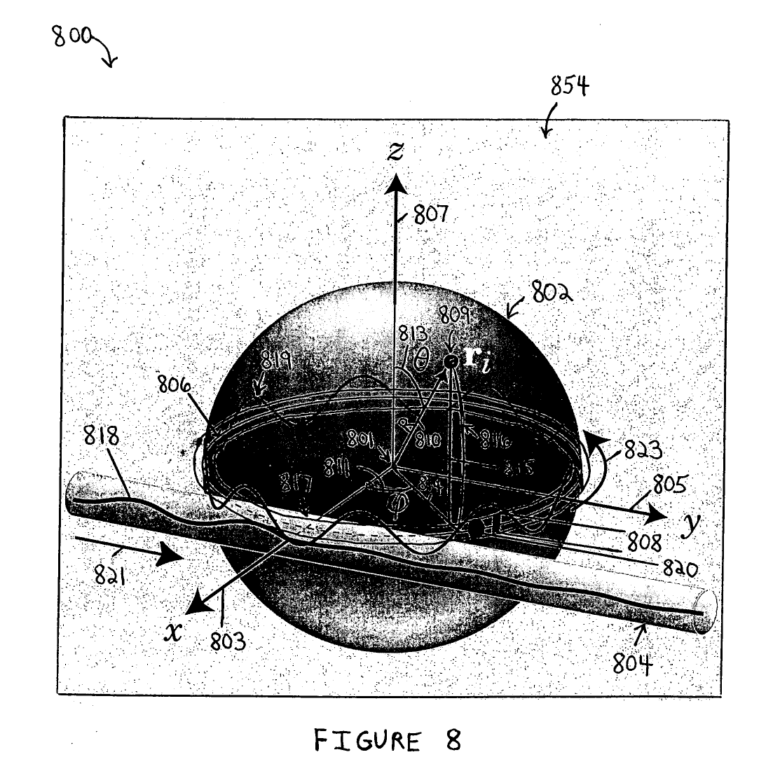

FIG. 8 is a more detailed illustration of an exemplary microsphere containment vessel 800 in order to identify specific novel features implemented in accordance with the invention and further explain the invention. Microsphere containment vessel 800 includes a microsphere 802 situated in an aqueous medium 854. The microsphere 802 with center 801 and radius R 810 is shown located at the center of a Cartesian coordinate system, with X axis 803, Y axis 805, and Z axis 807. An arbitrary position rᵢ 809 is located on the surface of the microsphere 802, defined by angle φ 811 from the X axis 803, angle θ 813 from the Z axis 807, projection in the XY plane 814, projection on the Z axis 815, and arc 816. Fiber 804 is eroded in region 817 providing an interface between the fiber 804 and the microsphere 802. Light signal 818 traveling in the direction of arrow 821 from laser 702 to photodiode 728 is coupled into a WGM 819 of the microsphere 802 and circulates in direction of arrow 823 about the equator 806 of microsphere 802. This coupling between fiber 804 and microsphere 802 results in changes in the transmitted signal 818 observable to the photo detector 728 as dips corresponding to resonant modes. Absorbed target entities 820 on the microsphere's surface will interact with the field of the WGM, polarize the molecule 820, and shift the frequencies of the modes. In accordance with the invention, a target receptor region 808 around the equator 806, determined to be a (extremely) sensitive region for adsorption of a single protein, has been selectively treated to promote adsorption, e.g., by silanizing the area. Single proteins absorbed in the target receptor region 808 surrounding the equator 806 produce detectable polarization changes which may be observed at photo detector 728 and the detected level of step change may be correlated to a specific protein. In FIG. 8, a single target entity 820, e.g., a single protein molecule, is shown adsorbed on the surface of the microsphere 802 in target region 808.

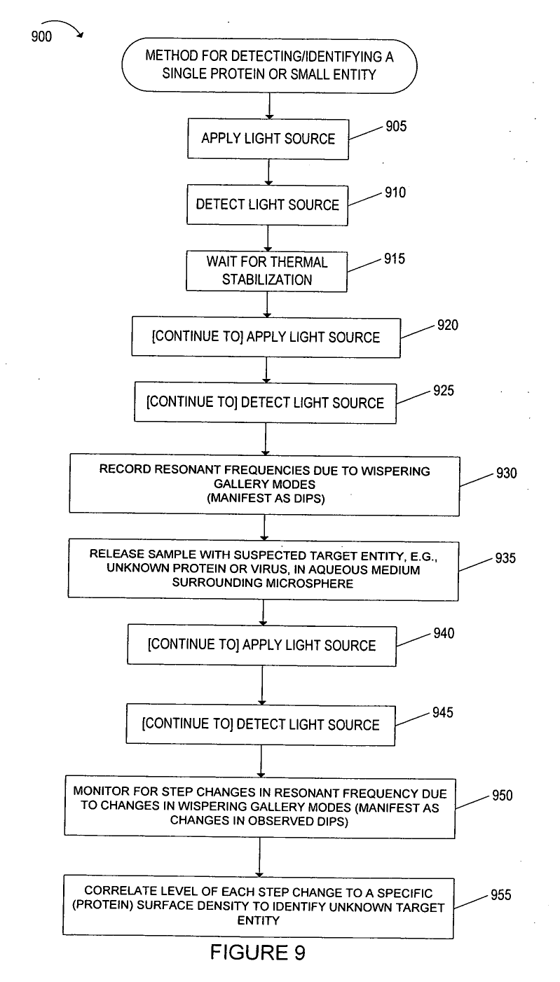

FIG. 9 is a flowchart 900 of an exemplary method that may be used, in accordance with the invention, to detect and/or identify a single protein, virus, or small entity. At step 905, under the control of the laser control routine 748, the tunable DFB laser 702 is turned on and set to the pre-selected frequency, e.g., about 400 nm. In step 910, the laser output is detected by photo detector 728, which generates a corresponding electrical signal. The electrical signal is forwarded to signal processing circuit 730, and the output of circuit 730 is used as laser feedback information by laser control routine 748 to regulate laser 702. In step 915, the temperature control routine 746 monitors sensors in temp control/monitoring device 718 and regulates heaters in device 718 to achieve thermal stabilization. In addition, during step 915, the laser control routine 748 is also monitoring for thermal stabilization via, e.g., frequency stabilization. After stabilization is achieved, operation proceeds to step 920. Step 920, indicates that the light source activated in step 905 remains on, and subsequent step 925 indicates that the monitoring initiated in step 910 remains active. Next, in step 930, the frequency shift measuring routine 750, processes signal output from detected signal processing circuit 730 and records resonant frequencies due to the whispering gallery modes of the microsphere, manifest as dips in the signal. When the frequency shift measuring routine 750 has determined that a stable baseline has been obtained, the operator may be prompted to release the sample with the target entity 720, into the aqueous medium 714 surrounding the microsphere 702. In step 935, the operator releases the sample with suspected target entity 720 through the target entity injection element 716 into aqueous medium 714. Step 940 indicates that the light source activated in step 905 continues to apply light. Target entity 720, e.g., a protein molecule, migrates through aqueous medium 714, and is adsorbed on the high sensitivity receptor region 708, resulting in a polarization level of the protein molecule that is significant enough to effect the WGMs and be detected by the photo detector 728 as a measurable frequency step level shift. Proceeding to step 945, the operation of the photo detector 728 has continued since activated in step 910. Next in step 950, signal changes, e.g. step shifts in resonant frequency due to changes in WGM, manifest as changes observed in the dips in transmitted signal are detected by circuit 730, and the information is forwarded to frequency shift measuring routine 750, where the level of the shift is determined by comparing the present information to the baseline (pre sample injection) information recorded in step 930. Next, in step 955, the target identification routine 752 is notified and compares the measured step change of step 950 to information included in the target database 744, to identify the unknown target entity 720 and/or determine concentration level information on the target entity 720.

Some or all of the steps of the method shown in flowchart 900 may be repeated on an ongoing basis.

Section 4: Alternatives

Further alternatives to the present invention, such as adjustments to the width of the equator belt, adjustments to the index of refraction (of the microsphere and/or medium), adjustments to the radius of the microsphere, and/or adjustments to the wavelength of the laser light can be made using the following observations. As will be appreciated, these parameters, as well as the desired level of frequency shift due to the adsorption of a single target entity molecule or particle, may be inter-related with one another and/or may depend on characteristics (such as the mass, the molecular surface density, the size, the excess polarizability) of the target entity molecule or particle of interest. Supporting theory and experimental observations are now described in detail. Recently Vollmer et al. (See, e.g., F. Vollmer, D. Braun, A. Libchaber, M. Khoshsima, I. Teraoka, S. Arnold, Appl. Phys. Lett. 80, 1 (2002). (incorporated herein by reference).) have reported specific detection of unlabelled biomolecules on a spherical surface (radius R≅0.15 mm), from the frequency shift of whispering gallery modes (WGMs). The modes were stimulated in a dielectric sphere immersed in an aqueous environment, by coupling light evanescently from an optical fiber. (See, e.g., A. Serpengüzel, S. Arnold, G. Griffel, Opt. Lett. 20, 654 (1995). (incorporated herein by reference).) The authors claim unprecedented sensitivity for the adsorption of protein molecules with spatial uniformity.

Using the observation described above, optical theory was used for describing this effect in an asymptotic limit (2πR/λ>>1). Then a comparison was made between the predicted size dependence with new experiments to confirm the theoretical explanation. Calculations were performed to gage the effect of reducing the size while placing protein molecules at specific locations on the sphere surface. The results obtained showed that for particular locations, the sensitivity for single protein adsorption can be enhanced by orders of magnitude.

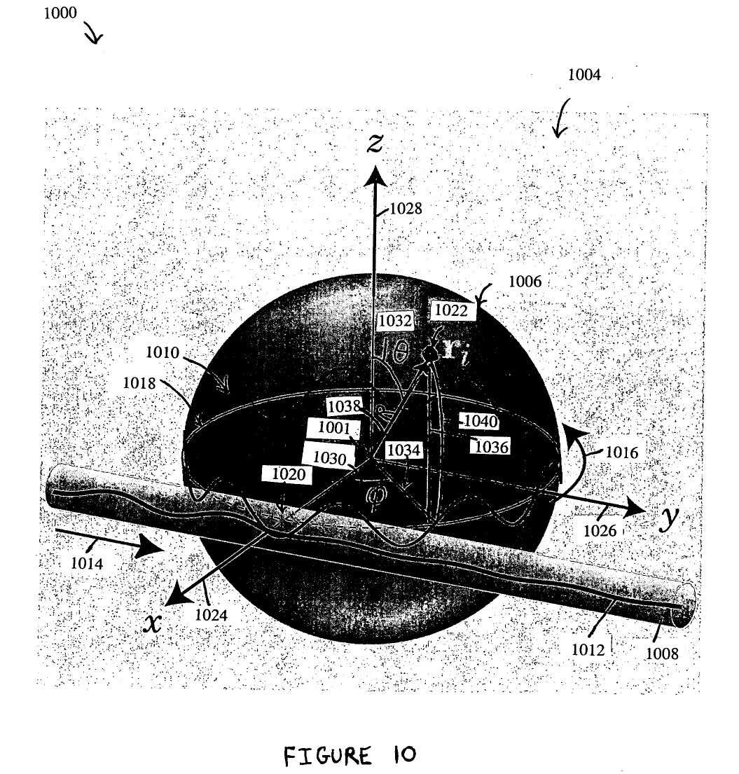

FIG. 10 shows a detailed illustration 1000 of a microsphere 1006 submerged in an aqueous medium 1004, e.g., water, and an optical fiber 1008. The microsphere 1006 with center 1001 and radius R 1038 is shown located at the center of a Cartesian coordinate system, with X axis 1024, Y axis 1026, and Z axis 1028. An arbitrary position rᵢ 1022 is located on the surface of the microsphere 1006, defined by angle φ 1030 from the X axis 1024, angle θ 1032 from the Z axis 1028, projection in the XY plane 1034, projection on the Z axis 1036, and arc 1040. Fiber 1008 is eroded in region 1020 providing an interface between the fiber 1008 and the microsphere 1006.

Light 1012 from a tunable DFB laser (not shown) is coupled into a WGM 1010 of the sphere 1006 from an eroded optical fiber (See, e.g., J. P. Laine, B. E. Little, H. A. Haus, IEEE Photonics Technol. Lett. 11,1429 (1999). (incorporated herein by reference).) 1008 and circulates, in direction 1016, about the equator 1018. Resonant modes are detected from dips in the transmission 1012 through the fiber 1008. A protein molecule diffuses to the sphere's surface from the surrounding aqueous medium 1004 and is adsorbed at position rᵢ 1022, where it interacts with the evanescent field of the WGM. The index i distinguishes each adsorbed protein molecule. This interaction polarizes the molecule, shifting the frequency of the mode.

To evaluate the shift δω in angular frequency ω for a single protein molecule, it is useful to consider the energy of interaction as a first-order perturbation to a single photon resonant state, with semi-classical field E₀(r)e^{iωt}. The evanescent tail of the field induces a dipole moment in the protein in excess of the displaced water, δpe^{iωt}, causing a shift in photon energy of the resonant state, [symbol] δω=−δp·E₀*(rᵢ)/2. The excess dipole moment can be represented in terms of the real part of an excess polarizability αₑₓ, i.e. δp=αₑₓE₀(rᵢ). The fractional frequency shift for a protein positioned at rᵢ is given by dividing the perturbation by the energy of the mode (i.e., [symbol] ω), as represented by integrating over the energy density in the interior,

$${( \frac{δ ω}{ω} )}_{i} ≅ \frac{- {α}_{ex} {\vert {E}_{0} ( {r}_{i} ) \vert}^{2}}{2 \int {ϵ}_{s} {\vert {E}_{0} ( r ) \vert}^{2} ⅆ V} . \qquad ( 1 )$$

The integral in the denominator is taken over the interior of the sphere 1006, which includes the overwhelming majority of the mode energy (>94%). (See, e.g., D. Q. Chowdhury, S. C. Hill, and M. M. Mazumder, IEEE J. Quant. Elec. 29, 2553 (1993). (incorporated herein by reference).) This approximation simplifies the analysis by allowing the homogeneous permittivity εₛ of the sphere to be pulled through the integral. The factor of 2 preceding this integral results from adding equal electric and magnetic contributions.

It should be noted that for protein molecules, which are composed of a variety of amino acids, αₑₓ is roughly proportional to the mass of the molecule, (See, e.g., Wen, T. Arakawa, and J. S. Philo, Anal. Biochem. 240, 155 (1996). (incorporated herein by reference).) and the shift in frequency in accordance with Eq. 1 should behave in the same way.

Equation 1 represents the shift due to an individual molecule at an arbitrary position on the sphere 1006, a point we will return to in calculating the optimal effect. However, in Ref. 1 (See, e.g., F. Vollmer, D. Braun, A. Libchaber, M. Khoshsima, I. Teraoka, S. Arnold, Appl. Phys. Lett. 80, 1 (2002). (incorporated herein by reference).) a large number of protein molecules are distributed over random locations on the sphere's surface. To account for each of these molecules we sum the singular contribution in Eq. 1 over N randomly located molecules and then turn this discrete sum into an integral over surface differentials;

$${\sum }_{i}^{N} {\vert {E}_{0} ( {r}_{i} ) \vert}^{2} ≅ {σ}_{p} \int {\vert {E}_{0} ( r ) \vert}^{2} ⅆ A ,$$

where σₚ, the protein surface density, is N/(4πR²). With this transformation from a discrete to continuous sum, Eq. 1 becomes

$$\frac{δ ω}{ω} ≅ - \frac{{α}_{ex} {σ}_{p}}{2 {ϵ}_{0} {ϵ}_{rs}} \frac{\int {\vert {E}_{0} ( r ) \vert}^{2} ⅆ A}{\int {\vert {E}_{0} ( r ) \vert}^{2} ⅆ V} , \qquad ( 2 )$$

where εₛ has been written in terms of a relative permittivity, εₛ=ε₀εᵣₛ.

We now evaluate Eq. 2 for a general TE mode for which the interior field at distance r from the sphere center is given as E₀=Aᵢₙjₗ(k₀r\sqrt{εᵣₛ})\hat{L}Yₗₘ, (See, e.g., J. D. Jackson, Classical Electrodynamics, 2nd edition, Wiley, New York (1975), p.745. (incorporated herein by reference).) where Aᵢₙ is the amplitude, jₗ(z) is a spherical Bessel function, \hat{L} is a dimensionless angular momentum operator (\hat{L}=−ir×Λ), k₀=ω/c with c being the speed of light in vacuum, and Yₗₘ is a spherical harmonic function. Fortunately both the surface and volume integrals in Eq. 2 contain precisely the same angular integrands. Consequently,

$$\frac{δ ω}{ω} ≅ - \frac{{α}_{ex} {σ}_{s}}{2 {ϵ}_{0} {ϵ}_{rs}} \frac{{[ {j}_{l} ( {k}_{0} R \sqrt{{ϵ}_{rs}} ) ]}^{2} {R}^{2}}{{\int }_{0}^{R} {[ {j}_{l} ( {k}_{0} r \sqrt{{ϵ}_{rs}} ) ]}^{2} {r}^{2} ⅆ r} , \qquad ( 3 )$$

where R is the radius of the sphere. On resonance, the volume integral in the denominator of Eq. 3 may be asymptotically (2πR/λ>>1) related to the surface value of jₜ² through

$${\int }_{0}^{R} {[ {j}_{l} ( {k}_{0} r \sqrt{{ϵ}_{rs}} ) ]}^{2} {r}^{2} ⅆ r ≅ {\frac{{R}^{3}}{2} [ {j}_{l} ( {k}_{0} R \sqrt{{ϵ}_{rs}} ) ]}^{2} \frac{{ϵ}_{rs} - {ϵ}_{rm}}{{ϵ}_{rs}} ,$$

where εᵣₘ is the relative permittivity of the surrounding medium. (See, e.g., C. C. Lam, P. T. Leung, K. Young, J. Opt. Soc. Am. B 9, 1585 (1992). (incorporated herein by reference).) Inserting this expression into Eq. 3, we find that the fractional frequency shift is given by a surprisingly simple formula,

$$\frac{δ ω}{ω} ≅ - \frac{{α}_{ex} {σ}_{p}}{{ϵ}_{0} ( {ϵ}_{rs} - {ϵ}_{rm} ) R} = - \frac{{α}_{ex} {σ}_{p}}{{ϵ}_{0} ( {n}_{s}^{2} - {n}_{m}^{2} ) R} . \qquad ( 4 )$$

where nₛ and nₘ are the refractive indices of the sphere and aqueous medium, respectively. The analysis of δω/ω for TM modes involves changing the field in Eq. 2. The result produced by a similar analysis has the same αₑₓσₚ/R dependence with numerically calculated shifts that only differ from the TE shifts by a few percent, for our silica-water interface.

The 1/R size dependence in Eq. 4 is expected for a homogeneous sphere. If such a sphere accretes a layer δR thick, it must preserve the product k₀R for a given resonance, and consequently δk₀/k₀=δω/ω=−δR/R. However, the formula becomes more complicated when the sphere is optically heterogeneous, as revealed in Eq. 4. Nonetheless, when the surface is saturated with protein, as revealed by no additional shift regardless of the external concentration, a plot of −δω/ω vs. 1/R will have a slope δR_{eff}, the effective thickness of the layer. It should be noted that δR_{eff} as defined can be negative, if the adsorbed material has a polarizability less than that of an equal volume of water. This odd circumstance is not the case for protein adsorption, since the optical permittivity of proteins is higher than that of water. In fact proteins have permittivities close to that of quartz.

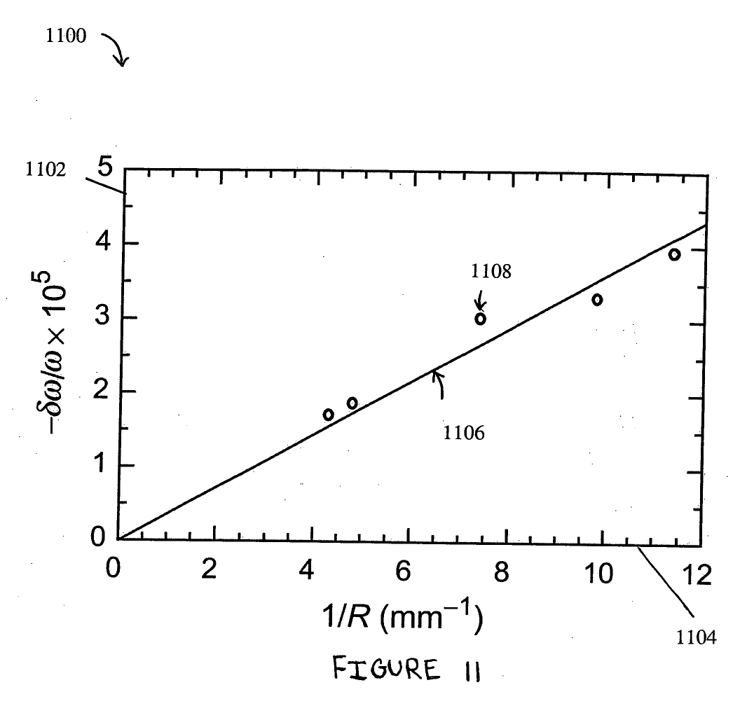

We have performed experiments on the adsorption of BSA protein on quartz microspheres. The silica glass surface is sensitized for protein adsorption by chemical modification with vapor phase 3-aminopropyltriethoxysilane following oxygen plasma cleaning. (See, e.g., K. H. Choi, J. P. Bourgoin, S. Auvay, D. Esteve, G. S. Duesberg, S. Roth, M. Burghard, Surf. Sci. 462, 195 (2000). (incorporated herein by reference).) The graph 1100 of FIG. 11 shows δω/ω×10⁵ on the vertical axis 1102, 1/R (mm⁻¹) on the horizontal axis 1104, individual experimental measured sample data points 1108, represented by small circles, and a linear fit to the data represented by line 1106. In FIG. 11, the resonance shifts −δω/ω are measured for complete saturation, using a current tuned DFB laser (See, e.g., G. Griffel, S. Arnold, D. Taskent, A. Serpenguezel, J. Connolly, D. G. Morris, Opt. Lett. 21, 695 (1996) (incorporated herein by reference).) operating at a nominal wavelength of 1.34 μm and are shown as a function of 1/R. Protein injection was implemented only after equilibrium was reached at 23° C. The system was verified to have returned to this temperature when wavelength shift measurement was taken. The spheres 1006 ranged in radius, R 1038, from 88 μm to 232 μm (412<2πR/λ<1087). Within the scatter in the data over this size range, a 1/R size dependence appears reasonable. The slope of the fit of line 1106 is δR_{eff}=3.6 nm.

An effective thickness of 3.6 nm is very close to the least dimension of BSA as revealed through x-ray crystallography. (See, e.g., D. C. Carter, X. M. He, S. H. Munson, P. D. Twigg, K. M. Gernert, M. B. Broom, T. Y. Miller, Science 244, 1195 (1989). (incorporated herein by reference).) BSA resembles a thick pancake with a heart-shaped profile; the least dimension is the height of the pancake. Furthermore, from the effective thickness and Eq. 4, it is possible to estimate the molecular surface density, σₚ=δR_{eff}ε₀(nₛ²−nₘ²)/αₑₓ. We calculate this surface density using the excess polarizability arrived at from differential refractive index measurements (See, e.g., F. Vollmer, D. Braun, A. Libchaber, M. Khoshsima, I. Teraoka, S. Arnold, Appl. Phys. Lett. 80, 1 (2002). (incorporated herein by reference).) [αₑₓ=4πε₀(3.85×10⁻²¹ cm³)], and the usual refractive indices for quartz and water, with the result σₚ=2.9×10¹² cm⁻². So a BSA molecule occupies an area σₚ⁻¹=3.4×10⁻¹³ cm². This agrees well again with crystallographic data, for which the area of the heart-shaped projection is 3.7×10⁻¹³ cm². It appears that BSA forms an extremely compact layer on the microsphere surface.

Single protein detection would be possible by looking at steps in the change of δω/ω with time, and this in turn provides a possible means for separately measuring αₑₓ. Since the light within a WGM 610 circumnavigates the equator 618 (θ=π/2) in an orbit which is confined to a thin ring, molecules at polar angles outside the ring cannot influence the mode frequency. The greatest signal comes from molecules which stick at θ=π/2. For a TE mode which circulates at the equator l=m, and the angular intensity is proportional to |\hat{L}Yₗₗ|², which for large l is proportional to |Yₗₗ|². (See, e.g., J. D. Jackson, Classical Electrodynamics, Wiley, New York (1962), p. 753. (incorporated herein by reference).) So the ratio of the frequency shift for a protein at the equator to that averaged over random positions on the surface is enhanced by a factor EF=4π|Yₗₗ(π/2,φ)|². This spatial enhancement EF can be significant. For the average size particle used in FIG. 11, l˜1000 and EF≅36. To obtain the average shift for an individual protein at a random position, we set the surface density in Eq. 4 to σ=1/(4πR²) with the result (δω/ω)ᵣ=−αₑₓ/[4πε₀(nₛ²−nₘ²)R³]. The shift due to a single protein at the equator is (δω/ω)ₑ=EF×(δω/ω)ᵣ, or

$${( δ ω / ω )}_{e} = - \frac{{α}_{ex} {\vert {Y}_{ll} ( π / 2 , ϕ ) \vert}^{2}}{{ϵ}_{0} ( {n}_{s}^{2} - {n}_{m}^{2} ) {R}^{3}} . \qquad ( 5 )$$

This single protein shift has a large size dependence. Since |Yₗₗ(π/2,φ)|² increases roughly in proportion to l^{1/2} or R^{1/2}, the single protein shift should go as R^{-5/2}. Currently, we can detect a fractional frequency change as small as 10⁻⁸. Since we can see a shift of one fiftieth of a line width, this requires that the Q be 2×10⁶. This Q is controlled by overtone vibrational absorption of water at 1.34 μm and the size of the microsphere. Leakage at the quartz-water interface limits the smallest radius for which this sensitivity is reasonable to approximately 50 μm. For a first-order TE mode within such a particle, and for a wavelength of 1.34 μm, 4π|Yₗₗ(π/2,φ)|²=20.8. Under these conditions, the smallest detectable single protein polarizability α_{sd}=4πε₀(2.4×10⁻¹⁷ cm³), or 6230 times the polarizability of BSA. Protein masses seldom exceed 10⁶ Da, which is only 15 times the mass of BSA. Thus using known microsphere systems, single protein measurements are not readily detectable from the resonance shift at 1.34 μm. The problem may be overcome by working in the frequency region for blue light where water absorption is reduced by more than a factor of 100, and by choosing a material for the microsphere with a larger refractive index. In accordance with one novel feature of the invention, the frequency of the light selected for use in the microsphere sensor may bein the blue region of the spectrum. The wavelength λ may be selected to match the characteristics of the microsphere. A reduction in the size of the wavelength λ may correspond to a reduction in the size of the microsphere. In accordance with another novel feature of the invention, the material selected for the microsphere of the invention has a larger refractive index than those chosen in known embodiments of the microsphere sensor.

As an example of an embodiment of the invention, a microsphere of amorphous sapphire has a refractive index of 1.7 at a wavelength of approximately 400 nm (blue diode laser with external cavity), which enables the radius to be reduced to approximately 3.6 μm for a Q of 2×10⁷ in water. Assuming the ability to see, e.g., detect, a shift of a fiftieth of a linewidth as before, the least measurable fractional shift would be 10⁻⁹. The minimum detectable polarizability projected from Eq. 5 is now approximately three times the polarizability of BSA, a number which is consistent with large protein molecules such as thyroglobulin, ferritin and virus particles (e.g. lambda phage). Adsorption onto the equator may be promoted by selectively silanizing the equator.

Although the invention has been described with respect to proteins, the invention can be used with other small entities, molecules, etc.

What is claimed is:

1. A method for determining the presence or concentration of a substance in a medium, the method comprising:
a) providing a sensor in the medium, wherein the sensor includes at least one optical carrier and a microsphere having a surface including receptors for the substance, wherein the receptors are provided substantially at a belt surface area including an equator of the microsphere, and wherein surface areas of the microsphere other than the belt surface area are substantially free of receptors, each of the at least one optical carrier being coupled with the microsphere;
b) applying a light source to one of the at least one optical carriers of the sensor;
c) detecting light from one of the at least one optical carriers of the sensor; and
d) determining a presence or concentration of the substance based on a property of the detected light, wherein the property is based on a shift in resonance of the microsphere.

2. The method of claim 1 wherein the light source emits light at a wavelength λ, wherein the microsphere has a radius R and a refractive index n, and wherein an arclength width of the belt is substantially the square root of Rλ/2πm.

3. The method of claim 1 wherein the microsphere is formed of a material having an index of refraction in water of approximately 1.7.

4. The method of claim 1 wherein the microsphere has a radius in a range of 3.6-10 μm.

5. The method of claim 1 wherein each of the at least one optical carriers of the sensor are optically coupled with the microsphere at the equator.

6. The method of claim 1 wherein the microsphere is formed of amorphous sapphire.

7. The method of claim 1 wherein light source is controlled to emit light in the blue spectrum.

8. The method of claim 7 wherein light source is controlled to emit light at about 400 nm.

9. The method of claim 1 wherein the shift in resonance of the microsphere is detectable when any of the receptors in the belt surface area capture a single molecule having a mass of about 200,000 Da.

10. A system for determining the presence or concentration of a substance in a medium, the system comprising:
a) a sensor, for immersion in the medium, the sensor including
i) at least one optical carrier, and
ii) a microsphere having a surface including receptors for the substance, wherein the receptors are provided substantially at a belt surface area including an equator of the microsphere, and wherein surface areas of the microsphere other than the belt surface area are substantially free of receptors, each of the at least one optical carrier being coupled with the microsphere;
b) a light source for applying light to one of the at least one optical carriers of the sensor;
c) a detector for detecting light from one of the at least one optical carriers of the sensor; and
d) means for determining a presence or concentration of the substance based on a property of the detected light, wherein the property is based on a shift in resonance of the microsphere.

11. The system of claim 10 wherein the light source emits light at a wavelength λ, wherein the microsphere has a radius R and a refractive index n, and wherein an arclength width of the belt is substantially the square root of Rλ/2πn.

12. The system of claim 10 wherein the microsphere is formed of a material having an index of refraction in water of approximately 1.7.

13. The system of claim 10 wherein the microsphere has a radius in a range of 3.6-10 μm.

14. The system of claim 10 wherein each of the at least one optical carriers of the sensor are optically coupled with the microsphere at the equator.

15. The system of claim 10 wherein the microsphere is formed of amorphous sapphire.

16. The system of claim 10 wherein light source is controlled to emit light in the blue spectrum.

17. The system of claim 16 wherein light source is controlled to emit light at about 400 nm.

18. The system of claim 10 wherein the shift in resonance of the microsphere is detectable by the detector when any of the receptors in the belt surface area capture a single molecule having a mass of about 200,000 Da.

19. For use in a system including a light source, and a light detector, for determining the presence or concentration of a substance in a medium, a sensor comprising:
a) at least one optical fiber;
b) at least one microsphere, the at least one microsphere
i) being coupled with the optical fiber,
ii) having a surface including receptors for the substance, wherein the receptors are provided substantially at a belt surface area including an equator of the microsphere, and wherein surface areas of the microsphere other than the belt surface area are substantially free of receptors, each of the at least one optical carrier being coupled with the microsphere,
wherein, when light is applied to the optical fiber, a resonance within the microsphere is excited,
wherein, if the substance adsorbs to the receptors on the microsphere surface, a shift in the resonance occurs, and
wherein a presence or concentration of the substance can be determined based on the shift in resonance.

20. The sensor of claim 19 wherein the substance is a protein, and
wherein the receptors are complementary amines.

20. The sensor of claim 19 wherein the substance is a virus particle, and wherein the receptors are complementary to the virus particle.

21. The sensor of claim 19 wherein the substance is DNA, and wherein the receptors are complementary to the DNA.

22. The sensor of claim 19 wherein the microsphere is formed of a material having an index of refraction in water of approximately 1.7.

23. The sensor of claim 19 wherein the microsphere has a radius in a range of 3.6-10 μm.

24. The sensor of claim 19 wherein each of the at least one optical fibers of the sensor are optically coupled with the microsphere at the equator.

25. The sensor of claim 19 wherein the microsphere is formed of amorphous sapphire.

26. A method for fabricating a sensor for determining the presence or concentration of a substance in a medium, the method comprising:
a) optically coupling at least one microsphere and an at least one optical core; and
b) providing a surface of the microsphere with a receptor complementary to the substance, wherein the receptors are provided substantially at a belt surface area including an equator of the microsphere, and wherein surface areas of the microsphere other than the belt surface area are substantially free of receptors.

27. The method of claim 26 wherein the act of providing a surface of the microsphere with a receptor complementary to the substance, wherein the receptors are provided substantially at a belt including an equator of the microsphere, and wherein surface areas of the microsphere other than the belt surface area are substantially free of receptors includes
i) applying a binding agent to the surface of the microsphere;
ii) selectively establishing the receptors on the belt surface area by attaching receptors to the binding agent substantially at only the belt surface area.

28. The method of claim 27 wherein the act of providing a surface of the microsphere with a receptor complementary to the substance, wherein the receptors are provided substantially at a belt including an equator of the microsphere, and wherein surface areas of the microsphere other than the belt surface area are substantially free of receptors further includes
iii) making the surface areas of the microsphere other than the belt surface area non-reactive.

29. The method of claim 27 wherein the act of applying a binding agent to the surface of the microsphere includes coating the microsphere with 2-(3-4 epoxycyclohexyl) ethyltrimethoxysilane.

30. The method of claim 27 wherein the act of selectively establishing the receptors on the belt surface area by attaching receptors to the binding agent substantially at only the belt surface area includes
A) placing the microsphere in a solution of amines or a vapor including amines, and
B) irradiating the belt surface area with UV light.

31. The method of claim 30 wherein the amines are ammonia ethylenediamine.

32. The method of claim 30 wherein the optical core is optically coupled with the equator of the microsphere, and
wherein the act of irradiating the belt surface area with UV light is performed by providing UV light to the optical core.

33. The method of claim 28 wherein the act of making the surface areas of the microsphere other than the belt surface area non-reactive includes
A) placing the microsphere in a solution of mono-secondary amines or a vapor including mono-secondary amines, and
B) irradiating the microsphere with UV light.

34. The method of claim 28 wherein the act of making the surface areas of the microsphere other than the belt surface area non-reactive includes
A) placing the microsphere in a solution of mono-secondary amines or a vapor including mono-secondary amines, and
B) heating the microsphere.

35. The method of claim 26 wherein the act of optically coupling at least one microsphere and an at least one optical core is performed such that the at least one optical core is optically coupled with the equator of the at least one microsphere.
